(19) United States
(12) Patent Application Publication
(10) Pub. No.: US 2021/0005302 A1
(43) Pub. Date: Jan. 7, 2021

(54) SYSTEM AND METHOD FOR MANAGING OFF-LABEL DRUG USE WITHIN A HEALTH CARE NETWORK

(71) Applicant: Patientory, Inc., Atlanta, GA (US)

(72) Inventor: Chrissa Tanelia McFarlane, Atlanta, GA (US)

(21) Appl. No.: 16/583,130

(22) Filed: Sep. 25, 2019

Related U.S. Application Data
(60) Provisional application No. 62/736,363, filed on Sep. 25, 2018.

(51) Int. Cl. G16H 20/10; G16H 10/60; H04L 9/06; H04L 9/30; G06F 21/62

(52) U.S. Cl. CPC G16H 20/10; G16H 10/60; G06F 16/27; H04L 9/30; G06F 21/6218; H04L 9/0637

(57) ABSTRACT

A system and method for managing off-label drug use within a health care network is disclosed herein. The system includes a Health Information Exchange system for managing off-label drug use within a health care network. The health information exchange system may comprise one or more user interfaces. The one or more user interfaces may be accessed by one or more users via one or more user devices. Additionally, the health information exchange system may be connected with a user device and a provider device through a communication network. Furthermore, the system may comprise a group of components for managing off-label drug use within the health care network. The group of components may include a processor, interface(s), and a memory. The memory may include an off-label base module and an off-label safety module. The off-label base module may include a permission module, a correlation generator module, and a recommendation module.

### OTHER RELATED APPLICATIONS

The present application is a U.S. Non-Provisional Patent Application claiming priority of U.S. Provisional Patent Application Ser. No. 62/736,363 filed on Sep. 25, 2018, which is hereby incorporated by reference.

### BACKGROUND OF THE INVENTION

### Field of the Invention

The present disclosure is generally related to a health care network, and more particularly related to a method for managing off-label drug use within the health care network.

### Description of the Related Art

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also correspond to implementations of the claimed technology.

To protect important information, utilizing storage on cloud networks is one approach to provide data redundancy. For sensitive information, the information may be stored in an encrypted form. Blockchain leverages both cloud networks and encryption to define storage of all information in a block wise manner. The blocks can be added to the blockchain in a linear and chronological order. The blockchain helps to store and track data in a secured manner. Further, the blockchain can be used in various fields such as gaming and gambling, diamond industry, real estate, medical industry, or e-voting.

Prescription drugs are marketed in the U.S. generally, carry a unique, FDA approved label. Prescription drugs are often prescribed for uses other than what the FDA has approved. As such, off-label drugs can be medications used in a manner not specified in the FDA's approved packaging label, or insert. Currently, usage of the off-label drug is not systematic and not managed properly, and thus leads to a cumbersome task of managing the off-label drug use. Therefore, there is a need for an improved method for managing the off-label drug use that may be efficient, profitable, and robust.

Applicant believes that a related reference corresponds to U.S. Patent No. US20040078220A1 issued for a System and method for collection, distribution, and use of information in connection with health care delivery. However, the reference differs from the present invention because it fails to address the issue of providing a secure and efficient method for storing sensitive information such as off-label drugs that do not include a unique FDA approved label. Additionally, the present invention utilizes storage on cloud networks for the safe keeping of sensitive information. The present invention addresses these issues by providing an efficient method and system for managing off-label drug use with a health care network.

Other documents describing the closest subject matter provide for a number of more or less complicated features that fail to solve the problem in an efficient and economical way. None of these patents suggest the novel features of the present invention.

### SUMMARY OF THE INVENTION

It is one of the objects of the present invention to provide a system and method for managing off-label drug use within a health care network that efficiently manages off-label drug use.

It is another object of this invention to provide a system and method for managing off-label drug use within a health care network that stores information in an efficient encrypted form to ensure the safety of the sensitive information stored therein.

It is still another object of the present invention to provide a system and method for managing off-label drug use within a health care network that allows health care practitioners to efficiently observe the usage of off-label drugs.

It is yet another object of this invention to provide such a device that is inexpensive to implement and maintain while retaining its effectiveness.

Further objects of the invention will be brought out in the following part of the specification, wherein detailed description is for the purpose of fully disclosing the invention without placing limitations thereon.

### BRIEF DESCRIPTION OF THE DRAWINGS

With the above and other related objects in view, the invention consists in the details of construction and combination of parts as will be more fully understood from the following description, when read in conjunction with the accompanying drawings in which:

FIG. 1 illustrates a network connection diagram of a Health Information Exchange (HIE) system for managing off-label drug use within a heath care network, according to various embodiments.

FIG. 2 illustrates a method for symmetric encryption of data, according to various embodiments.

FIG. 2A illustrates a method for asymmetric encryption of data, according to various embodiments.

FIG. 3 illustrates a method for hybrid encryption of data, according to various embodiments.

FIG. 4 illustrates a system for storing and accessing data in the health care network, according to various embodiments.

FIG. 5 illustrates a system for storing and accessing data in the health care network implemented specifically over a blockchain network, according to various embodiments.

FIG. 6 illustrates a flowchart showing an example method performed by an off-label base module, according to various embodiments.

FIG. 7 illustrates a flowchart showing an example method performed by a permission module, according to various embodiments.

FIG. 8 illustrates a flowchart showing an example method performed by a correlation generator module, according to various embodiments.

FIG. 9 illustrates a flowchart showing an example method performed by a recommendation module, according to various embodiments.

FIG. 10 illustrates a flowchart showing an example method performed by a user module, according to various embodiments.

FIG. 11 illustrates a flowchart showing an example method performed by a provider access module, according to various embodiments.

FIG. 12 illustrates a flowchart showing an example method performed by a research access module, according to various embodiments.

### DETAILED DESCRIPTION OF THE EMBODIMENTS OF THE INVENTION

Some embodiments of this disclosure, illustrating all its features, will now be discussed in detail. The words “comprising,” “having,” “containing,” and “including,” and other forms thereof, are intended to be open ended in that an item or items following any one of these words is not meant to be an exhaustive listing of such item or items, or meant to be limited to only the listed item or items.

It should also be noted that as used herein and in the appended claims, the singular forms “a,” “an,” and “the” include plural references unless the context clearly dictates otherwise. Although any systems and methods similar or equivalent to those described herein can be used in the practice or testing of various embodiments of the present disclosure, various embodiments of the systems and methods will be described.

Embodiments of the present disclosure will be described more fully hereinafter with reference to the accompanying drawings in which like may numerals represent like elements throughout the several figures, and in which various example embodiments are shown. Various embodiments may, however, be embodied in many different forms and should not be construed as limited to the embodiments set forth herein. The examples set forth herein are non-limiting examples and are merely examples among other possible examples.

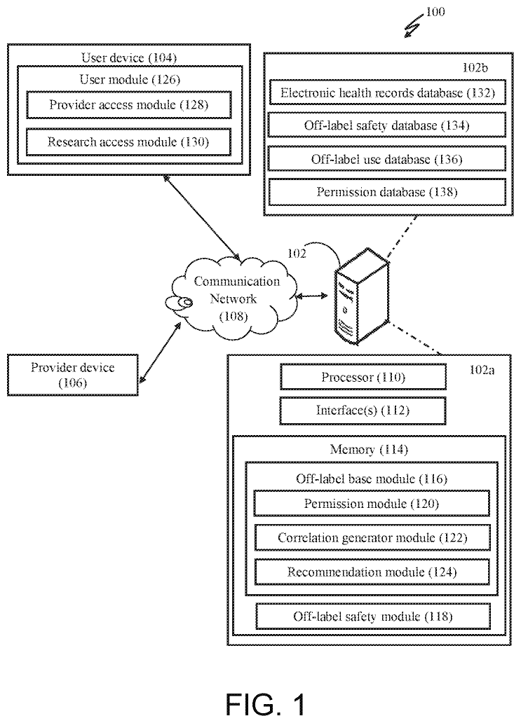

FIG. 1 illustrates a network connection diagram 100 of a Health Information Exchange (HIE) system 102 for managing off-label drug use within a health care network. The HIE system 102 may comprise one or more user interfaces. The one or more user interfaces may be accessed by one or more users via one or more user devices 104. The HIE system 102 may be connected with a user device 104 and a provider device 106, through a communication network 108.

The communication network 108 may be a wired and/or a wireless network. The communication network 108, if wireless, may be implemented using communication techniques such as Visible Light Communication (VLC), Worldwide Interoperability for Microwave Access (WiMAX), Long Term Evolution (LTE), Wireless Local Area Network (WLAN), Infrared (IR) communication, Public Switched Telephone Network (PSTN), Radio waves, and other communication techniques known in the art.

The HIE system 102 may comprise a group of components 102a for managing off-label drug use within the health care network. The group of components 102a may include a processor 110, interface(s) 112, and a memory 114. The memory 114 may include an off-label base module 116 and an off-label safety module 118. The off-label base module 116 may include a permission module 120, a correlation generator module 122, and a recommendation module 124.

The processor 110 may execute an algorithm stored in the memory 114 for managing the off-label drug use within the health care network. The processor 110 may also be configured to decode and execute any instructions received from one or more other electronic devices or server(s). The processor 110 may include one or more general purpose processors (e.g., microprocessors) and/or one or more special purpose processors (e.g., digital signal processors (DSPs), System On Chips (SOCs),

Field Programmable Gate Arrays (FPGAs), or Application-Specific Integrated Circuits (ASICs)). The processor 110 may be configured to execute one or more computer-readable program instructions, such as program instructions to carry out any of the functions described in this description.

The interface(s) 112 may help an operator to interact with the HIE system 102. The interface(s) 112 may either accept inputs from users or provide outputs to the users, or may perform both the actions. In various embodiments, a user can interact with the interface(s) 112 using one or more user-interactive objects and devices. The user-interactive objects and devices may comprise user input buttons, switches, knobs, levers, keys, trackballs, touchpads, cameras, microphones, motion sensors, heat sensors, inertial sensors, touch sensors, or any combination of the above. Further, the interface(s) 112 may be implemented as a Command Line Interface (CLI), a Graphical User Interface (GUI), a voice interface, or a web-based user-interface.

The memory 114 may include, but is not limited to, fixed (hard) drives, magnetic tape, floppy diskettes, optical disks, Compact Disc Read-Only Memories (CD-ROMs), and magneto-optical disks, semiconductor memories, such as ROMs, Random Access Memories (RAMs), Programmable Read-Only Memories (PROMs), Erasable PROMs (EPROMs), Electrically Erasable PROMs (EEPROMs), flash memory, magnetic or optical cards, or other type of media/machine-readable medium suitable for storing electronic instructions. The memory 114 may comprise modules implemented as a program. In various embodiments, the memory 114 may comprise the off-label base module 116 and the off-label safety module 118. The off-label base module 116 may include the permission module 120, the correlation generator module 122, and the recommendation module 124.

In various embodiments, several users may interact with the HIE system 102, using the user device 104. The user device 104 may include a user module 126. The user module 126 may include a provider access module 128 and a research access module 130. Although a single user device has been illustrated, several user devices could similarly be connected to the communication network 108. Further, each of the user devices may have a device ID. In various embodiments, the device ID may be a unique identification code such as an International Mobile Equipment Identity (IMEI) code or a product serial number. It should be noted that a user may use a single user device or multiple user devices. Further, multiple users may use a single user device or multiple user devices. Further, the one or more users may receive and/or provide healthcare related products and services. The one or more users may include, for example and not limited to, patients, family and friends of the patients, hospitals, physicians, nurses, specialists, pharmacies, medical laboratories, testing centers, insurance companies, or Emergency Medical Technician (EMT) services.

The user device 104 may be a stationary device, a portable device, or a device accessed remotely. The user device 104 may be, but not limited to, a computer, a laptop, a tablet, a mobile phone, a smartphone, or a smart watch. In various embodiments, the user device 104 may include an imaging device that may be configured to capture a visual graphical element. The visual graphical element may include, for example but not limited to, a barcode, text, a picture, or any other forms of graphical authentication indicia. In various embodiments, the barcode may be one-dimensional or two-dimensional. Further, the imaging device may include a hardware and/or software element. In various embodiments, the imaging device may be a hardware camera sensor that may be operably coupled to the user device 104. In various embodiments, the hardware camera sensor may be embedded in the user device 104. In various embodiments, the imaging device may be located external to the user device 104. In various embodiments, the imaging device may be connected to the user device 104 wirelessly or via a cable. It should be noted that image data of the visual graphical element may be transmitted to the user device 104 via the communication network 108.

In various embodiments, the imaging device may be controlled by applications and/or software(s) configured to scan a visual graphical code. In various embodiments, a camera may be configured to scan a QR code. Further, the applications and/or software(s) may be configured to activate the camera present in the user device 104 to scan the QR code. In various embodiments, the camera may be controlled by a processor natively embedded in the user device 104. In various embodiments, the imaging device may include a screen capturing software (for example, screenshot) that may be configured to capture and/or scan the QR code on a screen of the user device 104.

In various embodiments, the provider device 106 may be used by a provider. In various embodiments, the provider device 106 may be operated, for example and not by limitation, by a hospital, an insurer, Contract Research Organizations (CROs), a drug company, or a pharmaceutical company. Further, the provider device 106 may communicate with a health record network through an interface. The provider device 106 may be, for example, a desktop, a smart phone, tablet, and a phablet.

In various embodiments, a group of databases 102b may be connected to the HIE system 102. In various embodiments, the group of databases 102b may be implemented over a blockchain network (such as a PTOYNet blockchain network or a PTOYNet Ethereum™ Blockchain network), and may be present as different databases installed at different locations. The group of databases 102b may include an electronic health records database 132, an off-label safety database 134, an off-label use database 136, and a permission database 138. The group of databases 102b may be configured to store data belonging to different users and data required for functioning of the HIE system 102. Different databases can be used in accordance with various embodiments; however, a single database may also be used for storing the data. Usage of the different databases may also allow segregated storage of different data and may thus reduce time to access desired data. In various embodiments, the data may be encrypted, time-dependent, piece-wise, and may be present as subsets of data belonging to each user. In various embodiments, the data may represent the results of one medical test in a series of multiple medical tests.

In various embodiments, the group of databases 102b may operate collectively or individually. Further, the group of databases 102b may store data as tables, objects, or other structures. Further, the group of databases 102b may be configured to store data required or processed by the HIE system 102. The data may include, but not limited to, a patient medical history, medical charts, medications, prescriptions, immunizations, test results, allergies, insurance provider, or billing information. Further, the data may be time-dependent and piece-wise. Further, the data may represent a subset of data for each patient. In various embodiments, the data may represent results of a medical test in a series of multiple medical tests. Further, the data may be securely stored. In various embodiments, the data may be encrypted.

In various embodiments, information stored in the group of databases 102b may be accessed based on users' identities and/or the users' authorities. The users' identities may be verified in one or more ways such as, but not limited to, biometric authentication (or bio-authentication), password or PIN information, user device registrations, a second-level authentication, or a third-level authentication. In various embodiments, the users' identities may be verified by the HIE system 102.

Information provided by the users in a real-time may be used, by the HIE system 102, to confirm the users' identities. In various embodiments, the users' identities may be verified using a name, a password, one or security questions, or a combination thereof. In various embodiments, a user may be identified using an encryption key and/or a decryption key.

In various embodiments, the data stored in the group of databases 102b may be accessed at different levels, for example using a first level subsystem and a second level subsystem. In various embodiments, a user may directly access the first level subsystem. To access data stored in the second level subsystem, the second level subsystem may need to be accessed through the first level subsystem. It should be noted that the communication between the first level subsystem and the second level sub-system may be encrypted. In various embodiments, the second level subsystem may be implemented over a blockchain network (such as a PTOYNet blockchain network). In various embodiments, the PTOYNet blockchain network may be used to implement smart contracts.

In various embodiments, a primary care physician may input data into the HIE system 102 using the user device 104. The data may be processed by the first level subsystem and the second level subsystem. The data may be stored on the first level subsystem and/or the second level subsystem of the HIE system 102. The data may include, but not limited to, one or more instructions to a patient to see a physician specialist. Further, the data may be stored in one or more blockchains of the second level subsystem. The patient may be able to access the data relating to the patient's care provided by the primary care physician. The patient may be able to retrieve the data using the user device 104 of the patient.

In accordance with various embodiments, the patient may communicate with the physician specialist using the HIE system 102. It should be noted that the physician specialist may be able to access the data of the patient from the first level subsystem and/or the second level subsystem. Further, the physician specialist may be able to communicate with the patient. It should be noted that some, all (or substantially all) communications between the primary care physician, the physician specialist and the patient may be stored and may be accessible on a blockchain network.

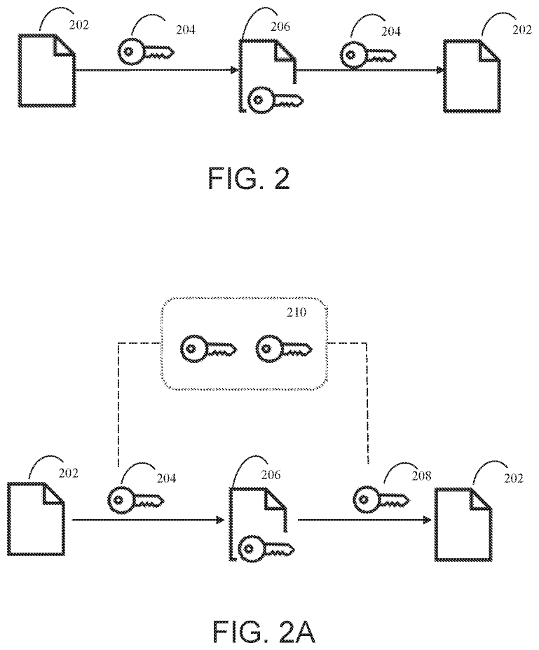

FIG. 2 illustrates a method for symmetric encryption of data, according to various embodiments. Original data 202 may be encrypted using a key 204 to obtain an encrypted data 206. The encrypted data 206 may be decrypted using the key 204 to obtain back the original data 202. It should be noted that encryption and decryption of the data may be performed using a same key. Further, one or more parties involved in a communication may have the same key to encrypt and decrypt the data.

FIG. 2A illustrates a method for asymmetric encryption of data, according to various embodiments. Original data 202 may be encrypted using a key 204 to obtain encrypted data 206. The encrypted data 206 may be decrypted using another key 208 to obtain the original data 202. It should be noted that encryption and decryption of the data may be performed using different keys, e.g., a key pair 210.

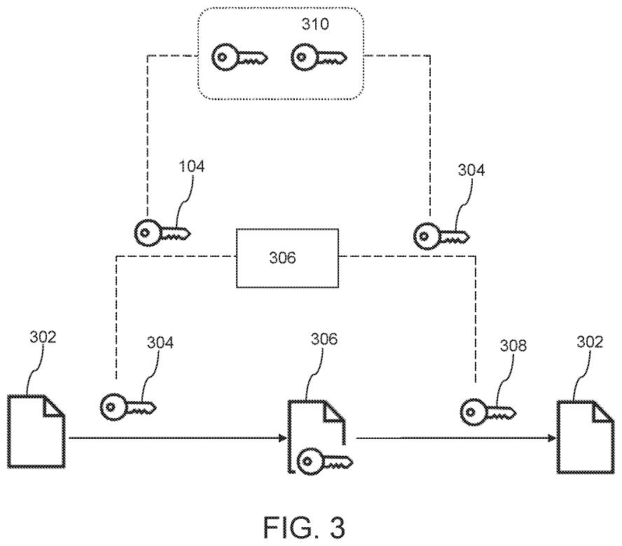

FIG. 3 illustrates a method for hybrid encryption of data, according to various embodiments. Both symmetric encryption and asymmetric encryption techniques may be used in tandem. In various embodiments, the symmetric encryption technique may be used to encrypt data 302 using a symmetric key 304 for producing encrypted data 306. The encrypted data 306 may be decrypted using another symmetric key 308 for obtaining data 302 (or back data). Further, a public key 310 may be used to encrypt the symmetric key 304 and a private key 312 may be used to encrypt the symmetric key 308, stored as an encrypted key 314. The public key 310 and the private key 312 may form a key pair 316.

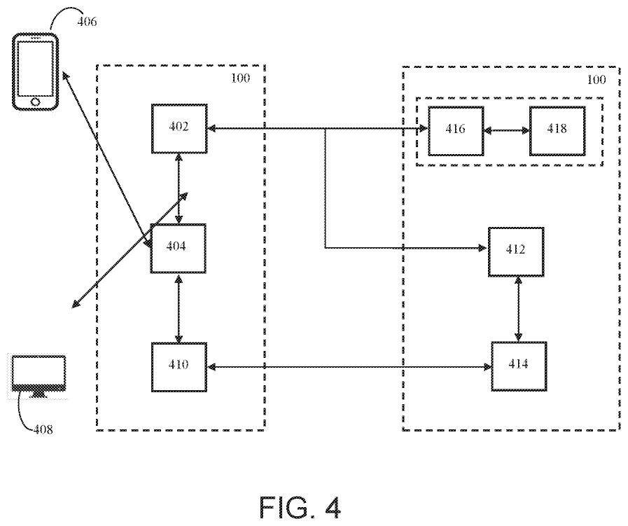

In accordance with various embodiments, FIG. 4 illustrates a system for storing and accessing data in the health care network, the first level subsystem may include a core service component 402 and a Remote Procedure Call (RPC) component 404. In various embodiments, the second level subsystem may include a blockchain node component 406 (e.g., quorum blockchain node component 406). In various embodiments, the first level subsystem may include the core service component 402, and the second level subsystem may include the RPC component 404 and the quorum blockchain node component 406. Further, the core service component 402 of the first level subsystem may be present in communication with third-party servers and databases of a hospital computing network 408. The hospital computing network 408 may include an Interplanetary File System (IPFS) module 410, an EHR synchronization service 412, and a blockchain node 414 (e.g., quorum blockchain node 414). Further, the IPFS module 410 may include an IPFS manager 416 and an IPFS node 418. The quorum blockchain node component 406 of the second level subsystem may communicate with the quorum blockchain node 414 of the hospital computing network 408. Patients may access the health care network for storing data through the user device 104, and a representative of a hospital may access the health care network through another user device.

In accordance with various embodiments, the representative of the hospital may want to synchronize Electronic Health Record (EHR) data of a patient. The first level subsystem and the second level subsystem may ask the patient for permission to allow a representative of the hospital to store the EHR data of the patient, through the IPFS module 410. Based at least on the permission granted by the patient, a signed transaction may be created to confirm the permission of the hospital to store the EHR data. Further, the signed transaction may activate a smart contract that may add hospital identification information such as a blockchain address to a list of permitted users.

In accordance with various embodiments, the signed transaction may be transmitted from the user device to the RPC component 404 of the first level subsystem and/or the second level subsystem. The RPC component 404 may communicate the signed transaction to the quorum blockchain node component 406 of the second level subsystem. The quorum blockchain node component 406 may activate one or more smart contracts. Thereafter, the quorum blockchain node component 406 may revise a state of one or more blockchains.

In accordance with various embodiments, based at least on the permission granted by the patient, the EHR synchronization service may obtain a list of patients from the RPC component 404. Further, the EHR synchronization service may confirm whether the patient has granted permission. Based at least on the permission, the first level subsystem and the second level subsystem may obtain the EHR data and may calculate a hash function for the EHR data. The HIE system 102 may match the hash function of the EHR data with a hash function for the patient blockchain on the quorum blockchain node component 406 of the second level subsystem. If the hash function of the EHR data matches with the hash function for the patient blockchain on the quorum blockchain node component 406 of the second level subsystem, the EHR data of the patient may remain unchanged.

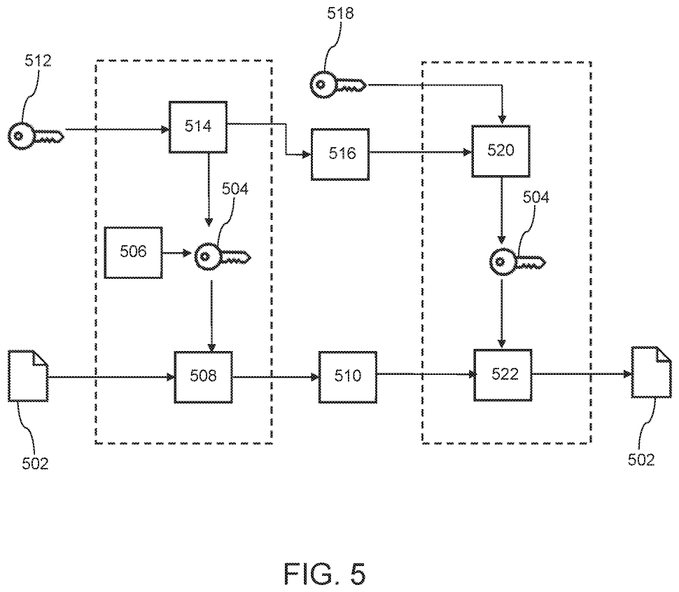

In accordance with various embodiments, FIG. 5 illustrates an example of a system for storing and accessing data in the health care network implemented specifically over a blockchain network (such as a PTOYNet blockchain network or a PTOYNet Ethereum™ Blockchain network), the HIE system 102 may execute an application for determining permission from the user for obtaining EHR data 502. In various embodiments, if the user grants the permission, the HIE system 102 may obtain the EHR data 502 for calculating a hash function for the EHR data 502. HIE system 102 may match the hash function of the EHR data 502 with a hash function for the user blockchain on the quorum blockchain node of the second level sub-system. In various embodiments, if the two hash matches, there is no change to the user's EHR data 502. In various embodiments, the two hash functions do not match, the HIE system 102 may generate a random string e.g., secret key 504, through a random key generator 506. The secret key 504 may be used for Advanced Encryption Standard (AES) encryption of the EHR data 502, in an AES encryptor 508, for generating encrypted EHR data 510.

In accordance with various embodiments, key 504 may then be encrypted by, for example, a Rivest-Shamir-Adleman (RSA) public key 512 of the patient, in an RSA encryptor 514, to generate an encrypted secret key 516. The HIE system 102 may also send the encrypted EHR data 510 to the core service component 402 for forwarding the data to the IPFS manager 416 of the hospital computing network 408 for storage. The IPFS manager 416 may send an IPFS hash function to the core service component 402 for further sending the IPFS hash function to EHR synchronization service 412. The EHR synchronization service 412 may further update the patient smart contract with the new IPFS hash function, the encrypted random key, a hash function of the unencrypted file, and file name.

In accordance with various embodiments, a hospital representative, such as a doctor or a hospital administration, may want to view the EHR data 502. In such a scenario, the user may first send a signed transaction to the RPC component 404 for granting permission to the hospital representative to view the EHR data 502. Once the permission is granted, the signed transaction may be added to the quorum blockchain node 414 and a new smart contract will be created for a blockchain corresponding to the hospital representative. After adding the signed transaction, the hospital representative may be able to view the EHR data 502 of the user, on a device.

In various embodiments, in order to view the EHR data 502 on the device, the HIE system 102 may collect the encrypted EHR data 510 from the user's blockchain and may decrypt the encrypted EHR data 510 using patient's RSA private key 518. The HIE system 102 may decrypt the encrypted secret key 516, in an RSA decryptor 520, using RSA private key of the hospital representative. The encrypted EHR data 510 may be decrypted using the RSA public key 512 of the hospital representative, in an AES decryptor 522. Further, the HIE system 102 may load the decrypted EHR data 502 to the smart contract previously created for the hospital representative.

After loading the decrypted EHR data 502, the RPC component 404 may obtain the signed transaction from the patient's user device and transmit the signed transaction to the quorum blockchain node component 406 of the second level subsystem. The quorum blockchain node component 406 may confirm ownership of the signed transaction and may execute the smart contract for the hospital representative to view the user's data.

In various embodiments, the patient may decline permission for the hospital representative to have access to the EHR data 502. In such an example, the user, through a user device, may send a signed transaction revoking permission to the RPC component 404. The RPC component 404 may forward the signed transaction to the quorum blockchain node component 406 of the second level subsystem. The quorum blockchain node component 406 may confirm ownership of the signed transaction and may delete the smart contract previously created to allow the hospital representative to have access to the patient's EHR data 502.

Further, the HIE system 102 may comprise the health record network for an intermediary enabling sharing of user's medical records with providers. To enable sharing, the user may grant specific permissions to the providers for accessing parts of the user's medical records stored in a user database implemented over a blockchain network. The user may also grant specific permissions to modify the user's medical records in the user database. In various embodiments, the user may comprise of any users constituting a value chain, such as doctors, nurses etc. In various embodiments, the user may be remote doctors logging into the HIE system 102 or doctors present in hospitals.

In various embodiments, the electronic health records database 132 may be configured to store medical data of a user. Further, the electronic health records database 132 may be a distributed blockchain database. It should be noted that a blockchain may be a continuously growing list of records, called blocks, which may be linked and secured using cryptography. Each block may contain a cryptographic hash of the previous block, a timestamp and transaction data.

In various embodiments, the off-label safety database 134 may be configured to store safety statistics or efficacy statistics of off-label drugs. The safety statistics or efficacy statistics may be determined by the off-label safety module 118, based on an analysis of meta tagged electronic health record data. As noted above, off-label drugs may correspond to a medication that is being used in a manner not specified in the FDA's approved packaging label or insert. As such, prescription drugs may be prescribed for uses other than what the FDA has approved. It should be noted that the drugs may change and results of the drugs may be defined and stored.

In various embodiments, the off-label use database 136 may be configured to store instances detected in the electronic health records database 132, during which a drug may be utilized for an off-label use. Further, the off-label use database 136 may store provides contexts or situations in which that drug may be utilized off-label. Further, the off-label use database 136 may store a correlation coefficient that may indicate how closely a given set of context factors correlate to the off-label use of the drug.

In various embodiments, the permission database 138 may be configured to store the medical data of the user, which may be accessed by the provider. It should be noted that the medical data stored in the permission database 138 may be authorized by the user for sharing with the provider.

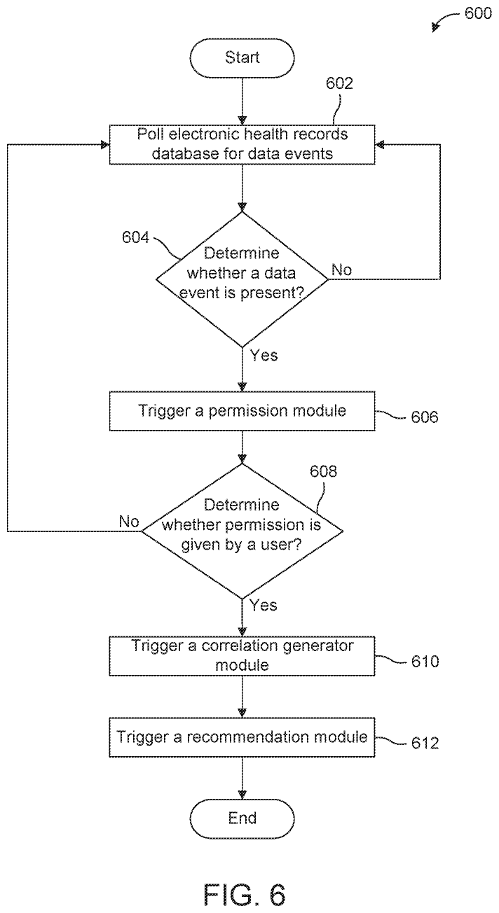

FIG. 6 illustrates an example flowchart 600 showing a method performed by the off-label base module 116, in accordance with various embodiments. An example process utilizing the off-label base module 116 will now be explained with reference to the flowchart 600 shown in FIG. 6. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The off-label base module 116 may poll electronic health records database 132 for data events, at step 602. The data events may be related to off-label drug use. It should be noted that the off-label drug use may include, for example use of pharmaceutical drugs for an unapproved indication or in an unapproved age group, dosage, or route of administration. Both prescription drugs and over-the-counter drugs (OTCs) may be used in off-label ways. Further, an ability to prescribe drugs for uses beyond officially approved indications may be used to good effect by the providers. For example, methotrexate can be used off-label due to immunomodulatory effects of the methotrexate. The immunomodulatory effects relieve various disorders.

Retiring to flowchart 600 of FIG. 6, off-label base module 116 may determine whether the data event is present, at step 604. In various embodiments, if the data event is not present, the off-label base module 116 may follow the step 602. In various embodiments, if the data event is present, the off-label base module 116 may trigger the permission module 120, a step 606.

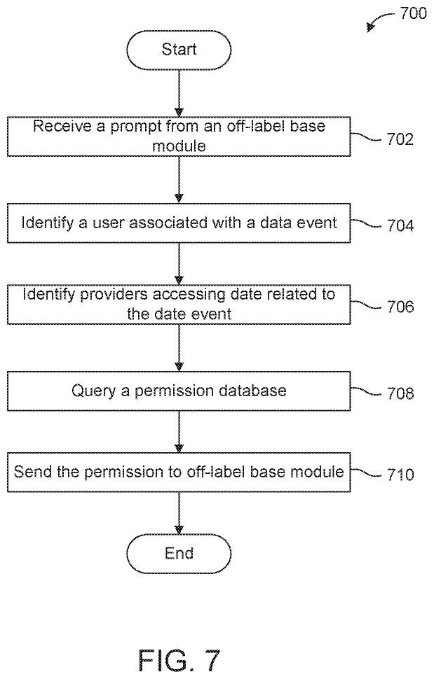

An example process utilizing the permission module 120 (triggered, for example at step 606 of flowchart 600 of FIG. 6) will now be explained with reference to the flowchart 700 shown in FIG. 7. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The permission module 120 may receive a prompt from the off-label base module 116, at step 702. The permission module 120 may identify a user associated with the data event, at step 704. The permission module 120 may identify providers accessing date related to the new date event, at step 706. In various embodiments, the hospital or CRO may have different permissions granted by the user than a pharmaceutical company. The permission module 120 may query the permission database 138, at step 708. The permission database 138 may be queried to determine what data, if any, the user associated with the data event is granted to the providers in question. The permission module 120 may send the permission to the off-label base module 116, at step 710. It should be noted that the permission may correspond to a permission level, that either grants no access to the data event, partial access to the data event, or full access to the data event.

Returning to FIG. 6, off-label base module 116 may determine whether permission is given by the user, at step 608. If, for example, the permission is not given, the off-label base module 116 may follow the step 602. If, for example, the permission is given, the off-label base module 116 may trigger the correlation generator module 122, at step 610. It should be noted that one or more levels of off-label trigger events, such as a major drug change in a procedure to a support drug change or a minor post-operative drug change. The trigger may be set based upon a group of drugs in a series i.e., the series of the drugs may change from a prescribed normal sequence. Further, the trigger may be set when a mix of drugs is not allowed and therefore a new off-label drug may be used.

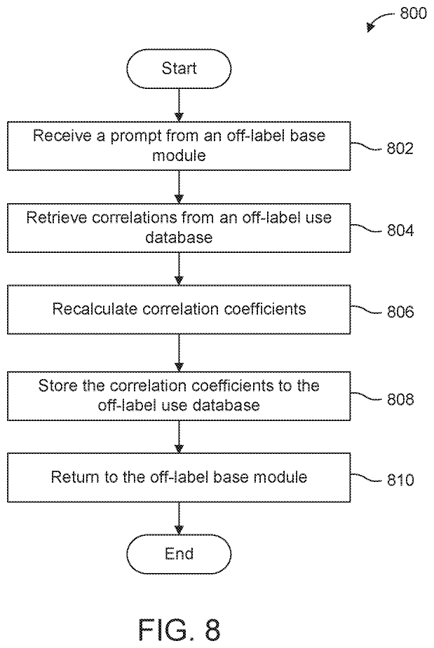

An example process utilizing the correlation generator module 122 (triggered, for example at step 610 of flowchart 600 of FIG. 6) will now be explained with reference to the flowchart 800 shown in FIG. 8. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The correlation generator module 122 may receive a prompt from the off-label base module 116, at step 802. The prompt may correspond to the data event. The correlation generator module 122 may retrieve correlations from the off-label use database 136, at step 804. It should be noted that current context of the data event may be identified and thereafter, the correlations related to the current context may be retrieved from the off-label use database 136. For example, Colchicine is indicated for treatment and prevention of gout. Further, the Colchicine is considered first-line treatment for acute pericarditis as well as preventing recurrent episodes. Further, the Colchicine has anti-inflammatory effect for pericarditis appears to be related to an ability to inhibit microtubule self-assembly, resulting in decreased leucocyte motility and phagocytosis. Other non-FDA-approved uses may include actinic keratosis, amyloidosis, Peyronie' s disease, and psoriasis.

For example, Propranolol is a non-selective beta-blocker used for the treatment of hypertension and the prophylaxis of angina pectoris. Further, a study shows that a single dose of propranolol immediately before the Scholastic Aptitude Test (SAT) significantly improved performance in high school students prone to cognitive dysfunction due to test anxiety. In addition to test taking, the propranolol has been tested for public speaking, performing surgery, musical recitals, and sports, all with varying degrees of benefit. Other off-label uses of the propranolol may include a treatment of thyroid storm, portal hypertension, and neuroleptic-induced akathisia.

Returning to flowchart 800 of FIG. 8, the correlation generator module 122 may recalculate correlation coefficients, at step 806. It should be noted that the correlations may be recalculated to incorporate values of the data events. The correlation generator module 122 may store the correlation coefficients to the off-label use database 136, at step 808. Thereafter, the correlation generator module 122 may return to the off-label base module 116, at step 810.

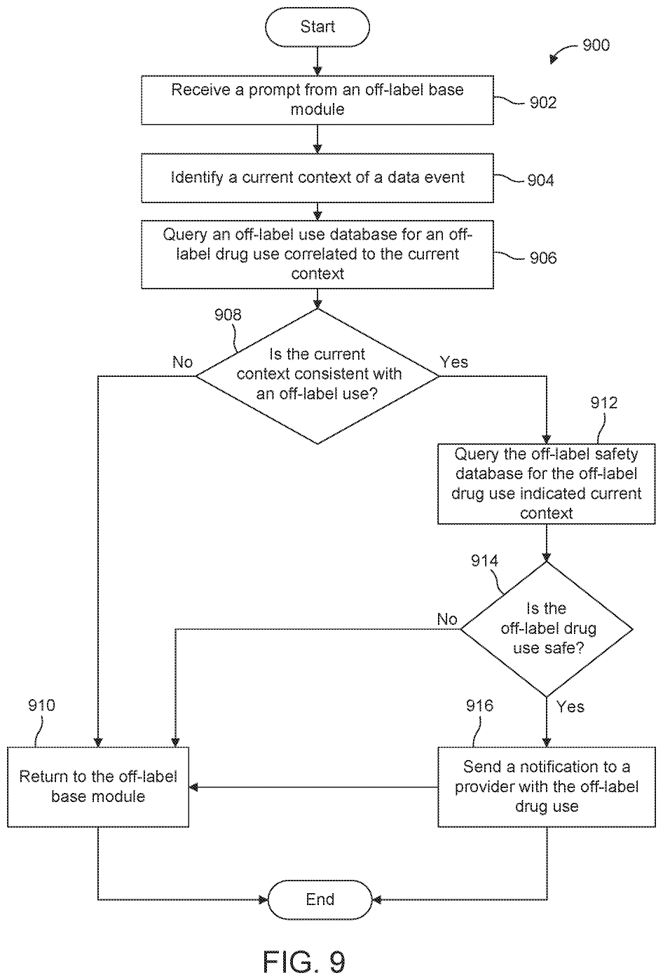

Returning to flowchart 600 of FIG. 6, the off-label base module 116 may trigger the recommendation module 124, at step 612. An example process utilizing the recommendation module 124 (triggered, for example at step 612 of flowchart 600 of FIG. 6) will now be explained with reference to the flowchart 900 shown in FIG. 9. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

Referring to FIG. 9, the recommendation module 124 may receive a prompt from the off-label base module 116, at step 902. The prompt may correspond to the data event. The recommendation module 124 may identify the current context of the data event, at step 904. For example, a woman having an age of 35 may be bleeding post-partum in an obgyn provider's facility. The recommendation module 124 may query the off-label use database 136 for the off-label drug use correlated to the current context, at step 906. In an example, a use of hemophilia drug is to stop bleeding in the current context. The recommendation module 124 may determine whether the current context is consistent with the off-label drug use, at step 908. If, for example, the current context is not consistent with the off-label drug use, the recommendation module 124 may return to the off-label base module 116, at step 910.

In an example, if a correlation coefficient of the off-label drug use to the current context is greater than a given threshold value, the recommendation module 124 may return to the off-label base module 116. For example case, the threshold value may be 0.70. If the current context has a correlation coefficient, for example, greater than the threshold value to the off-label drug use, the recommendation module 124 may query the off-label safety database 134 for the off-label drug use indicated current context, at step 912. The recommendation module 124 may determine whether the off-label drug use is safe, at step 914. If, for example, the off-label drug use is not safe or below the predefined threshold, the recommendation module 124 may follow the step 910.

On the other hand, if the off-label drug use is safe or above the predefined threshold, the recommendation module 124 may send a notification to a provider with the off-label drug use, at step 916. The recommendation module 124 may return to the off-label base module 116, at step 910. The notification may indicate a usage of the off-label drug in the context of the data event. Further, the recommendation module 124 may send statistics related to safety and frequency of the off-label drug use. It should be noted that the statistics related to the safety and frequency of the off-label drug use can be stored in the off-label safety database 134.

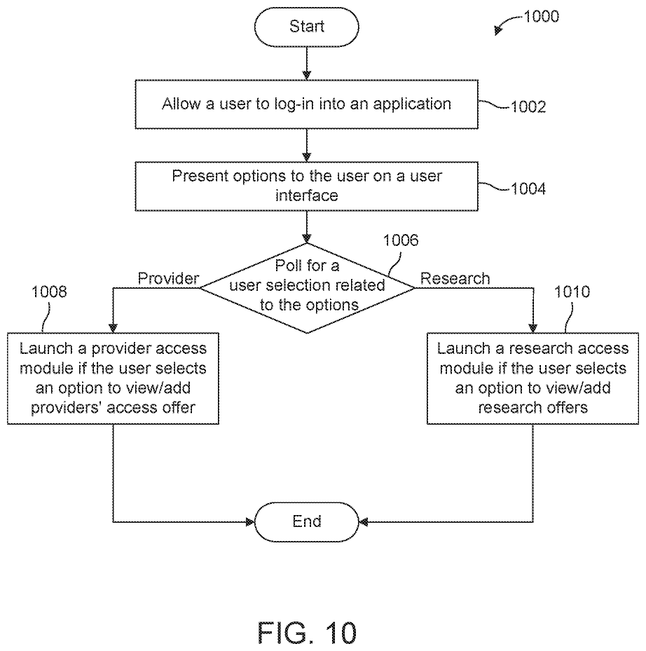

FIG. 10 illustrates an example flowchart 1000 showing a method performed by the user module 126, according to various embodiments. An example process utilizing of the user module 126 will now be explained with reference to the flowchart 1000 shown in FIG. 10. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

Referring to FIG. 10, the user module 126 may allow a user to log-in into an application, at step 1002. The application may reside in the user device 104. The user module 126 may present options to the user on the user interface, at step 1004. The options may be an option to view/add providers' access offers, and an option to view/add research offers. The user module 126 may poll for a user selection related to the options, at step 1006. If, for example, the user selects an option to view/add providers' access offer, the user module 126 may launch the provider access module 128, at step 1008.

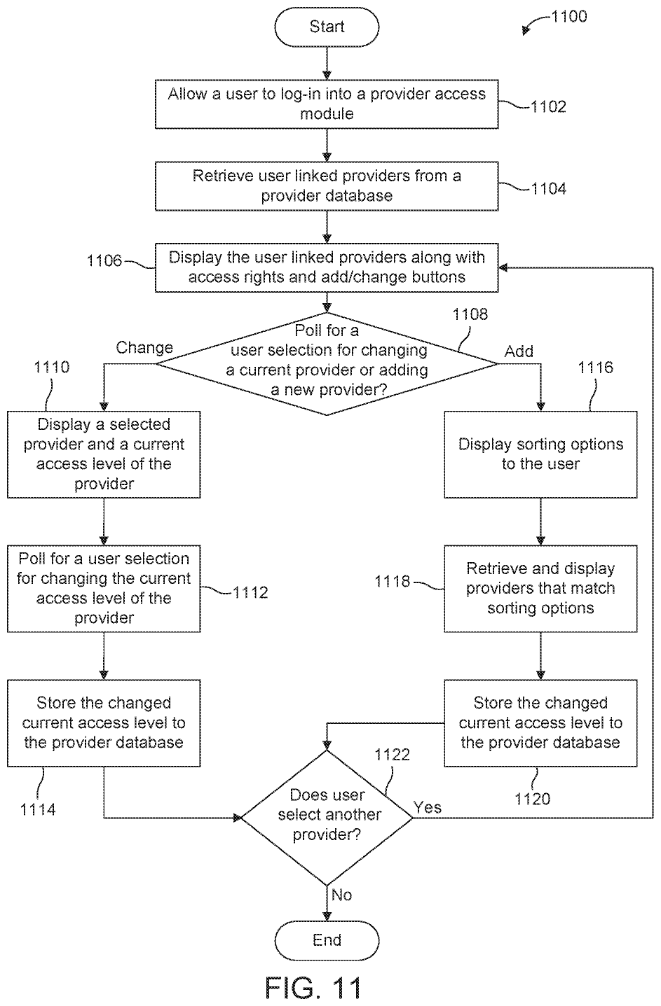

An example process utilizing of the provider access module 128 (launched for example at step 1008 of FIG. 10) will now be explained with reference to the flowchart 1100 shown in FIG. 11. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

Referring to FIG. 11, the provider access module 128 may allow the user to log-in, at step 1102. The provider access module 128 may retrieve user linked providers from a provider database, at step 1104. The user linked providers may correspond to a user has a data sharing relationship with the providers. The provider access module 128 may display the user linked providers along with access rights and add/change buttons, at step 1106. The user linked providers may be displayed on the user device 104 with the add/change buttons that allow the user to change access rights of currently linked providers and/or add a new provider. The provider access module 128 may poll for a user selection for changing a current provider or adding a new provider, at step 1108. If, for example, the user selects to change the current provider, the provider access module 128 may display a selected provider and a current access level of the provider, at step 1110. The current access level may correspond to an access to patient data. The provider access module 128 may poll for a user selection for changing the current access level of the provider, at step 1112. Thereafter, the provider access module 128 may store the changed current access level to the provider database, at step 1114.

Returning to step 1108, if the user selects to add the new provider, the provider access module 128 may display sorting options to the user, at step 1116. The sorting options may be a location or a type of the provider. The provider access module 128 may retrieve and display providers that match the sorting options, at step 1118. The sorting options may be retrieved from the provider database. Further, the providers matched with the sorting options may be displayed to the user. The provider access module 128 may store the changed current access level to the provider database, at step 1120. The provider access module 128 may then determine whether the user selects another provider, at 1122. For example, if the user selects another provider, then the provider access module 128 may follow the step 1106. Alternatively, if the user does not select another provider, then provider access module 128 may end the process.

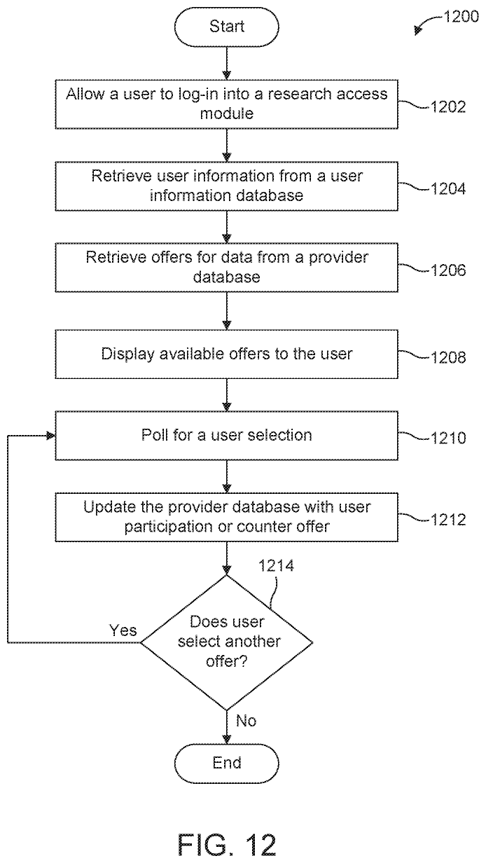

Returning to step 1006 of FIG. 10, if the user selects an option to view/add research offers, then user module 126 may launch the research access module 130, at step 1010. An example process utilizing the research access module 130 (launched for example at step 1010 of FIG. 10) will now be explained with reference to the flowchart 1200 shown in FIG. 12. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The research access module 130 may allow the user to log-in, at step 1202. The research access module 130 may retrieve user information from a user information database, at step 1204. The user information database may be located on the user device 104. The user information may be retrieved from a user device such as, for example, a glucometer or activity monitor. The research access module 130 may retrieve offers for data from the provider database, at step 1206. It should be noted that the offers for data that the user may provide to the CRO. The research access module 130 may display available offers to the user, at step 1208. The available offers that the user may fulfill. The research access module 130 may poll for a user selection, at step 1210. The research access module 130 may update the provider database with user participation or counter offer, at step 1212. It should be noted that the user may, for example, accept the current offer or indicate a price at which the user may provide the requested data.

Returning to FIG. 12, the research access module 130 may determine whether the user selects another offer, at step 1214. For example, if the user selects another offer, then the research access module 130 may follow the step 1210. On the other hand, if the user does not select offer, then the research access module 130 may end the process.

It will be appreciated that variants of the above disclosed, and other features and functions or alternatives thereof, may be combined into many other different systems or applications. Presently unforeseen or unanticipated alternatives, modifications, variations, or improvements therein may be subsequently made by those skilled in the art that are also intended to be encompassed by the following claims.

What is claimed is:

1. A computer-implemented method for managing an off-label drug use within a health care network, comprising:
a. retrieving one or more data events related to the off-label drug use, from an electronic health records database;
b. analyzing and identifying a context of the one or more data events;
c. correlating and matching the off-label drug use with a context of the one or more data events; and
d. providing a notification along with an information related to the off-label drug use to one or more providers based at least on a correlation level, and thereby managing the off-label drug use.

2. The computer-implemented method of claim 1, further comprising identifying a safe usage of the off-label drug based at least on a predefined threshold.

3. The computer-implemented method of claim 1, wherein the one or more providers include at least one individual belonging to a hospital, to an insurance company, to a contract research organization, or to a pharmaceutical company.

4. The computer-implemented method of claim 1, wherein sending a notification along with the information comprises providing at least one of statistics related to safety and frequency of the off-label drug use.

5. The computer-implemented method of claim 1, wherein retrieving one or more data events related to the off-label drug use comprises polling a health information exchange network for a relevant data event.

6. The computer-implemented method of claim 1, wherein retrieving one or more data events comprises accessing a blockchain database with a public key, wherein the blockchain database is supported by a health information exchange.

7. The computer-implemented method of claim 1, wherein identifying a context of the one or more data events comprises retrieving the context of the one or more data events from an off-label use database.

8. The computer-implemented method of claim 1, wherein correlating the off-label drug use with a context of the one or more data events comprises triggering a permission module to allow access to the context of the one or more data events in an off-label use database.

9. The computer-implemented method of claim 1, further comprising triggering a permission module, identifying a user associated with a data event, and providing a permission to access an off-label use database when the user is part of the health care network.

10. The computer-implemented method of claim 1, further comprising triggering a permission module, identifying one or more providers accessing data related to a data event, and providing a permission to access an off-label use database when at least one provider is part of the health care network.

11. A system for managing an off-label drug use within a health care network, comprising:
a. one or more processors; and
b. a memory storing instructions which, when executed by the one or more processors, cause the system to:
retrieve one or more data events related to the off-label drug use, from an electronic health records database;
identify a context of the one or more data events;
correlate the off-label drug use with a context of the one or more data events;
provide a notification along with an information related to the off-label drug use to one or more providers based at least on a correlation level, and thereby managing the off-label drug use; and
identify a safe usage of the off-label drug based at least on a predefined threshold.

12. The system of claim 11, wherein the one or more providers include at least one individual belonging to a hospital, to an insurance company, to a contract research organization, or to a pharmaceutical company.

13. The system of claim 11, wherein sending a notification along with the information comprises providing at least one of statistics related to safety and frequency of the off-label drug use.

14. The system of claim 11, wherein retrieving one or more data events related to the off-label drug use comprises polling a health information exchange network for a relevant data event.

15. The system of claim 11, wherein retrieving one or more data events comprises accessing a blockchain database with a public key, wherein the blockchain database is supported by a health information exchange.

16. The system of claim 11, wherein identifying a context of the one or more data events comprises retrieving the context of the one or more data events from an off-label use database.

17. A non-transitory, computer readable medium storing instructions which, when executed by a processor, cause a computer to perform a method for managing an off-label drug use within a health care network, the method comprising:
a. retrieving one or more data events related to the off-label drug use, from an electronic health records database;
b. triggering a permission module based on the one or more data events;
c. identifying a context of the one or more data events;
d. correlating the off-label drug use with a context of the one or more data events;
e. providing a notification along with an information related to the off-label drug use to one or more providers based at least on a correlation level, and thereby managing the off-label drug use; and
f. identifying a safe usage of the off-label drug based at least on a predefined threshold.

18. The non-transitory, computer readable medium of claim 17 wherein, in the method, correlating the off-label drug use with a context of the one or more data events comprises triggering a permission module to allow access to the context of the one or more data events in an off-label use database.

19. The non-transitory, computer readable medium of claim 17, wherein the method further comprises triggering a permission module, identifying a user associated with a data event, and providing a permission to access an off-label use database when the user is part of the health care network.

20. The non-transitory, computer readable medium of claim 17, wherein the method further comprises triggering a permission module, identifying one or more providers accessing data related to a data event, and providing a permission to access an off-label use database when at least one provider is part of the health care network.
